(19) United States
(12) Patent Application Publication
(10) Pub. No.: US 2020/0025764 A1
(43) Pub. Date: Jan. 23, 2020

(54) FOXC1 ANTIBODIES AND METHODS OF THEIR USE

(71) Applicant: JOHN WAYNE CANCER INSTITUTE, Santa Monica, CA (US)

(72) Inventor: Xiaojiang CUI, Los Angeles, CA (US)

(21) Appl. No.: 16/285,772

(22) Filed: Feb. 26, 2019

Related U.S. Application Data
(63) Continuation of application No. 15/293,117, filed on Oct. 13, 2016, now abandoned.
Application No. 15/293,117 is a continuation of application No. 14/250,148, filed on Apr. 10, 2014, now abandoned.
Application No. 14/250,148 is a continuation of application No. 13/366,170, filed on Feb. 3, 2012, now abandoned.
(60) Provisional application No. 61/439,825, filed on Feb. 4, 2011.

(51) Int. Cl. G01N 33/574; A61K 51/10; G01N 33/68; C07K 16/18; A61K 39/395; A61K 49/04; A61K 49/16

(52) U.S. Cl. CPC G01N 33/57415; A61K 51/1051; G01N 33/6872; C07K 16/18; A61K 2039/505; A61K 49/04; A61K 49/16; A61K 51/1093; C07K 2317/34; A61K 39/39533

(57) ABSTRACT

In one embodiment, an isolated antibody or functional fragment thereof which binds an antigenic peptide sequence of human FOXC1 is provided herein. Such antibodies or functional fragments may be used to diagnose, prognose or treat basal-like breast cancer. The antibody or functional fragment may be a monoclonal antibody produced by a hybridoma cell line. Thus, a hybridoma cell line that produces a FOXC1 monoclonal antibody which binds an antigenic peptide sequence of human FOXC1 as described above is also provided.

### PRIORITY CLAIM

This application is a continuation of U.S. patent application Ser. No. 15/293,117, filed Oct. 13, 2016 and now pending, which is a continuation of U.S. patent application Ser. No. 14/250,148, filed Apr. 10, 2014 and now abandoned, which is a continuation of U.S. patent application Ser. No. 13/366,170, filed Feb. 3, 2012 and now abandoned, which claims the benefit of U.S. Provisional Application No. 61/439,825, filed Feb. 4, 2011, all of which are hereby incorporated by reference as if fully set forth herein.

### REFERENCE TO SEQUENCE LISTING SUBMITTED VIA EFS-WEB

application Ser. No. 15/293,117 was filed via EFS-Web on Oct. 13, 2016 and includes an electronically submitted sequence listing in .txt format. The .txt file contains a sequence listing entitled “2016-10-13_Parent_Sequence_List_798778002US3.TXT” created on Oct. 13, 2016 and 5,787 bytes in size. The sequence listing contained in this .txt file is part of the specification and is hereby incorporated by reference herein in its entirety.

### BACKGROUND

Forkhead box transcription factors, including forkhead box C1 (FOXC1), are transcription factors characterized by a common 100-amino acid winged-helix DNA-binding domain termed the forkhead box domain, and play important roles in regulating the expression of genes involved in cell growth, survival, differentiation embryonic mesoderm development, migration, and longevity (Nishimura et al., 1998). As a result of the studies described herein, it has been determined that FOXC1 expression in human breast cancer, both at the mRNA and at the protein level, occurs consistently and exclusively in basal-like breast cancers (BLBC). Basal-like breast cancer (BLBC) has a poor prognosis and is often identified by a triple-negative phenotype (ER-/PR-/HER2-) and basal cytokeratins. Recently, however, overexpression of mRNA for the FOXC1 transcription factor has been identified as an important prognostic biomarker of BLBC (Ray et al. 2010). FOXC1 protein expression has also been found to be a significant predictor of overall survival on univariate and multivariate analyses (Ray et al. 2010).

Although many genes have been described to be characteristic biomarkers of certain cancer types, and many others are described to be of functional importance to the survival and maintenance of the malignant phenotype, very few are demonstrated to have robust prognostic significance. This is because very few are critical by themselves and instead are part of extremely large and complex networks of biomolecules whose overall function cannot be determined unless the molecules which are most central and pivotal in the network are identified. FOXC1 has been demonstrated to have high prognostic significance, being predictive of the high mortality and metastasis rate specifically associated with basal-like breast cancers. Thus, FOXC1 is an indicative and characteristic biomarker of BLBC. The clinical significance of FOXC1 as it relates to basal-like breast cancers is described in detail in International Patent Application No. PCT/US10/44817, filed Aug. 6, 2010, which is hereby incorporated by reference as if fully set forth herein.

The clinical significance of FOXC1 expression is not restricted to breast cancer but may extend to other cancers, including but not limited to, brain tumors (such as glioblastoma multiforme, or astrocytoma), colon cancer, gastric cancer, melanoma, neuroendocrine tumors, prostate cancer (e.g., androgen insensitive prostate cancer), renal cell cancer, sarcomas (such as synovial sarcoma), and leukemia. FOXC1 expression has been shown to define biologically and clinically aggressive subsets in such cancers and can be used both as a diagnostic as well as prognostic biomarker for these specific cancer types. Furthermore, FOXC1 is a suitable therapeutic target for BLBC and the other specific cancer types described above. Therefore, development of substances that target FOXC1 and are suitable for use in diagnosis, prognosis and treatment of BLBC and other cancers is desired.

The above described findings have clear and important implications for personalized medicine and personalized cancer care as detection of FOXC1 status of the described specific subsets of patients with breast cancer (and/or other cancers described above) will enable more tailored and specific therapeutic interventions with a greater likelihood of arresting disease progression, extending life expectancy or even achieving a cure.

### SUMMARY

In one embodiment, an isolated antibody or functional fragment thereof which binds an antigenic peptide sequence of human FOXC1 is provided herein. In some aspects, the antigenic peptide sequence may include amino acids 51-75 of human FOXC1 (SEQ ID NO:1). In another aspect, the antigenic peptide sequence is 5′-AHAEQYPGGMARAYGPYTPQPQPKD-3′ (SEQ ID NO:2) or 5′-C-AHAEQYPGGMARAYGPYTPQPQPKD-3′ (SEQ ID NO:3).

The antibody or functional fragment may be a monoclonal antibody produced by a hybridoma cell line. Thus, in one embodiment a hybridoma cell line that produces a FOXC1 monoclonal antibody which binds an antigenic peptide sequence of human FOXC1 as described above is provided.

In another embodiment, a method of diagnosing a basal-like breast cancer in a subject having breast cancer is provided. Such a method may include contacting one or more breast cancer cells with a FOXC1 antibody or functional fragment thereof conjugated to a diagnostic agent; detecting the presence or absence of FOXC1 in the one or more breast cancer cells; and diagnosing the subject as having a basal-like breast cancer when FOXC1 is present. The FOXC1 antibody or functional fragments thereof may be any which are described above and throughout the disclosure.

The diagnostic agent may be any suitable substance, including a radioactive substance, a dye or contrast agent, a fluorescent compound or molecule, a bioluminescent compound or molecule or an enzyme or enhancing agent.

Detecting the presence or absence of FOXC1 is accomplished by an in vitro immunoassay, such as immunocytochemistry (ICC), immunohistochemistry (IHC), Western blot or fluorescent in situ hybridization (FISH). Alternatively, an in vivo imaging modality may be used, such as magnetic resonance imaging (MRI), positron emission tomography (PET) or microPET, computed tomography (CT), PET/CT combination imager, cooled charged coupled device (CCD), camera optical imaging, optical imaging and single photon emission computed tomography (SPECT). When the presence or absence of FOXC1 is determined by an in vivo method, the FOXC1 antibody or functional fragment thereof should be conjugated to an intracellular delivery agent to facilitate deliver of the antibody or functional fragment thereof to the cytoplasm of target cells.

In another embodiment, a method of treating a basal-like breast cancer in a subject is provided. Such a method may include administering a therapeutically effective amount of pharmaceutical composition that comprises an intracellular delivery agent conjugated to a FOXC1 antibody or functional antibody fragment thereof. In some aspects, the intracellular delivery agent may be cell-penetrating peptide, a nanoparticle, a cationic lipid, poly-L-arginine, a liposomal drug or a polymeric carrier.

The antibody or functional fragment thereof binds an antigenic peptide sequence of human FOXC1, such as SEQ ID NO:2 or SEQ ID NO:3. Further, the FOXC1 antibody of functional fragment may also be conjugated to a therapeutic agent.

### BRIEF DESCRIPTION OF THE DRAWINGS

FIG. 1 is an immunoblot of MCF-7 human breast cancer cells that were transfected with either FOXC1(+) or vector (−) using polyclonal commercial FOXC1 antibodies (Santa Cruz Biotechnology) and FOXC1 monoclonal antibodies isolated from hybridoma cell line B2E3. Both antibodies were used at a 1:100 dilution.

FIG. 2 are representative images of MCF-7 human breast cancer cells transfected with GFP-FOXC1 (A) wherein immunofluorescence staining was performed using FOXC1 monoclonal antibodies isolated from hybridoma cell line B2E3 to detect successfully transfected cells (B). DAPI was used to stain the DNA of all cells (C).

FIG. 3 shows representative images of immunohistochemical staining of FOXC1 on two representative breast cancer samples (FOXC1 positive breast cancer tissue and FOXC1 negative breast cancer tissue) using FOXC1 monoclonal antibodies isolated from hybridoma cell line B2E3.

FIG. 4 is the sequence of human forkhead box protein Cl (FOXC1) (SEQ ID NO:1; Accession No. AAH70124). The target antigenic peptide sequence used to generate FOXC1 monoclonal antibodies according to some embodiments is in bold and underlined.

### DETAILED DESCRIPTION

The disclosure is directed to antibodies that specifically bind forkhead box protein Cl (FOXC1) and methods of their use. According to some embodiments described herein, such anti-FOXC1 antibodies may be used in methods to diagnose and treat basal-like breast cancer and were generated to develop a convenient, pragmatic, and efficient approach to immunohistochemical assays that exploit the diagnostic, prognostic and therapeutic potential of FOXC1 in breast cancer management.

An antibody as described herein is a molecule that includes an intact immunoglobulin or one or more portions of an immunoglobulin or immunoglobulin-related molecule that specifically binds to, or is immunologically reactive with an antigenic epitope of a target substance (e.g., a target protein). Such an immunoglobulin or immunoglobulin-related molecule may include monoclonal or polyclonal antibodies of any isotype (e.g., IgA, IgD, IgE, IgG or IgM), modified antibodies or functional antibody fragments.

The term modified antibody includes, but is not limited to genetically engineered or otherwise modified forms of immunoglobulins or functional fragments thereof, such as intrabodies, peptibodies, chimeric antibodies, fully human antibodies, humanized antibodies, bivalent or bispecific antibodies, and conjugate antibodies (e.g., diabodies, triabodies, tetrabodies). The modified antibodies may be monovalent or multivalent. The term functional antibody fragment includes one or more antigen binding fragments of antibodies alone or in combination with other molecules, including, but not limited to Fab′, F(ab′)₂, Fab, Fv, rlgG, scFv fragments, and single domain fragments. Examples of the types of antibodies, modified antibodies and functional antibody fragments that may be used in accordance with the embodiments described herein are further described in Huson P. J. & Souriau C., Engineered Antibodies, Nature Medicine (2003) 9(1):129-134, the subject matter of which is hereby incorporated by reference as if fully set forth herein.

FOXC1 Antibodies

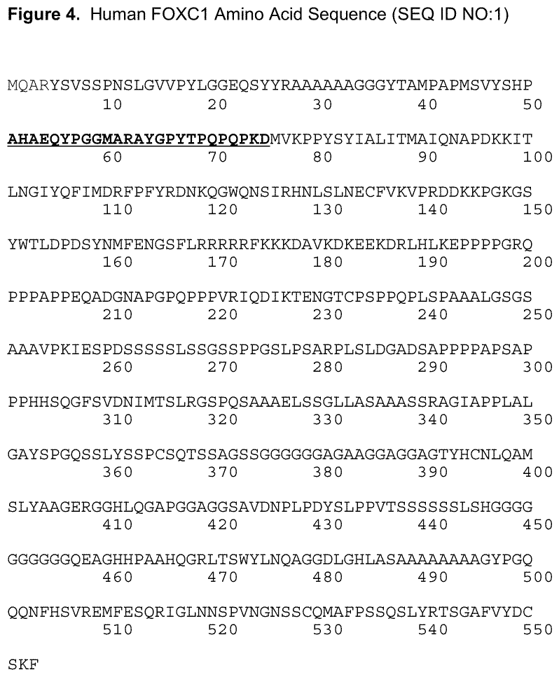

In some embodiments, the antibodies described herein specifically bind a target antigenic peptide sequence of human FOXC1 (FIG. 4; SEQ ID NO:1). In one aspect, the target antigenic peptide sequence corresponds to an epitope present in the intact, native state of the target protein, FOXC1. The target antigenic peptide sequence may correspond to a continuous epitope (i.e., composed of a contiguous sequence of amino acids in a protein) or a discontinuous epitope (i.e., a group of amino acids that are not contiguous but are brought together by folding of the peptide chain or by the juxtaposition of two separate polypeptide chains.

Several characteristics are important for selecting a suitable target antigenic peptide sequence within a target protein sequence such as FOXC1. Potential antigenic epitopes within a target protein sequence should have, but are not limited to, the following characteristics: (i) hydrophilic, because most naturally occurring proteins in aqueous solutions have hydrophilic residues on the surface and hydrophobic residues on the interior; (ii) surface-oriented, because antibodies and functional fragments thereof generally bind epitopes on the surface of proteins; and (iii) flexible, because it has been shown that epitopes have a high degree of mobility. Algorithms for predicting protein characteristics such as hydrophilicity/hydrophobicity and secondary structure regions such as alpha-helix, beta sheet and beta turn may aid selection of a potentially exposed, immunogenic internal sequence for antibody generation. A commercial software package may be used to assist with selecting a target antigenic peptide sequence, such as MacVector™, DNAStar™, and PC-Gene™. In one embodiment, the antibodies or functional antibody fragments bind a target antigenic peptide sequence that corresponds to the N-terminal transactivation domain of FOXC1. In one aspect, the target antigenic peptide sequence is 5′-AHAEQYPGGMARAYGPYTPQPQPKD-3′ (SEQ ID NO:2), which corresponds to amino acids 51 to 75 of SEQ ID NO:1 (see FIG. 4).

In some embodiments, a target antigenic peptide sequence may be conjugated to a carrier protein to enhance its immunogenicity. The carrier proteins may include epitopes that stimulate T-helper cells, which in turn help induce the B-cell response against the target antigenic peptide sequence. For example, the carrier peptide may include, but is not limited to, keyhole limpet hemacyanin (KLH), bovine serum albumin (BSA), ovalbumin (OVA) and rabbit serum albumin (RSA).

The conjugation of the target antigenic peptide sequence to the carrier protein may be accomplished by a suitable coupling method. In one aspect, the coupling method is a carbodiimide method, which uses cabodiimides that can activate the side chain carboxylic groups of aspartic and glutamic acid as well as the carboxyl terminal group to make them reactive sites for coupling with primary amines. In another aspect, gluteraldehyde may be used as a bifunctional coupling reagent that links two compounds through their amino groups. In another aspect, m-Maleimidobenzoyl-N-hydroxysuccinimide ester (MBS) can be used to link peptides to carrier proteins via cysteines. The coupling takes place with the thiol group of cysteine residues. If the chosen sequence does not contain Cys, a Cys residue may be placed at the N- or C-terminus to obtain controlled linking of the peptide to the carrier protein. Thus, in one embodiment, the target antigenic peptide sequence described above (SEQ ID NO:2; 5′-AHAEQYPGGMARAYGPYTPQPQPKD-3′) may have a cysteine added to the N-terminus to assist in its conjugation to a carrier protein as necessary (e.g., 5′-C-AHAEQYPGGMARAYGPYTPQPQPKD-3′; SEQ ID NO:3).

In some embodiments, the antibody that specifically binds a target antigenic peptide sequence of human FOXC1 (the “FOXC1 antibody” or the “FOXC1 antibodies”) is a monoclonal antibody. Monoclonal antibodies are highly specific because they are directed against a single antigenic site. Thus, monoclonal antibodies are useful to improve the selectivity and specificity of diagnostic and analytic assays as well as therapeutic applications as compared to conventional, polyclonal preparations.

In other embodiments, the FOXC1 antibody is a functional fragment of the monoclonal FOXC1 antibody as described above. The functional fragment of the monoclonal FOXC1 antibody may be a portion of the antibody (e.g., a domain), which retains the ability to specifically bind a target antigenic peptide sequence of human FOXC1. Examples of such antibody fragments include: F(ab′)₂, Fab′, Fab, single-chain Fv (hereinafter, referred to as “scFv”), disulfide-linked Fv (hereinafter, referred to as “dsFv”), a polymer thereof, dimeric V region (hereinafter, referred to as a “diabody”), and peptides or peptibodies containing complementarity determining regions (CDR).

Various methods known within the art may be used for the production of antibodies, including hybridoma, phage library and recombinant techniques (see, e.g., Antibodies: A Laboratory Manual, Harlow E. and Lane D, 1988, Cold Spring Harbor Laboratory Press, Cold Spring Harbor, N.Y.; and Making and Using Antibodies: A Practical Handbook, Howard G and Kaser M, 2006, CRC Press; both of which are hereby incorporated by reference as if fully set forth herein).

In one embodiment, the FOXC1 antibodies described herein are generated by a hybridoma cell line. The hybridoma cell line is produced by suitable hybridoma technique known in the art, such as those described by Kohler and Milstein (Kohler & Milstein, 1975). In a hybridoma technique, a host animal (e.g., a mouse or rabbit), is typically immunized with an immunizing agent to elicit lymphocytes that produce or are capable of producing antibodies that will specifically bind to the immunizing agent. Alternatively, the animal may be immunized with cells transfected with a vector containing a nucleic acid molecule encoding FOXC1 or an immunogenic fragment, derivative or variant thereof or the lymphocytes may be immunized in vitro. Such recombinant mAb production methods are is described further below.

In some embodiments, the immunizing agent may be a target protein antigen (e.g., FOXC1), a fragment thereof or a fusion protein thereof (e.g., the target antigenic peptide sequences of human FOXC1; SEQ ID NOs:2-3). The lymphocytes are then fused with an immortalized cell line (e.g., myeloma cells) using a suitable fusing agent, such as polyethylene glycol, to form a hybridoma cell. The hybridoma cells are then cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

The culture medium in which the hybridoma cells are cultured can then be assayed for the presence of monoclonal antibodies directed against the antigen. Preferably, the binding specificity of monoclonal antibodies produced by the hybridoma cells is determined by a suitable method known in the art, such as immunoprecipitation or an in vitro binding assay, such as radioimmunoassay (RIA) or enzyme-linked immunoabsorbent assay (ELISA).

After the desired hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by an immunoglobulin purification procedure known in the art including, but not limited to, protein A-Sepharose, hydroxylapetite chromatography, gel electrophoresis, dialysis, or affinity chromatography.

In other embodiments, antibodies may be made by recombinant DNA methods known in the art. Methods for recombinant production of monoclonal antibodies are described, for example in U.S. Pat. No. 4,816,397 to Boss et al. (expired) and U.S. Pat. No. 4,816,567 to Cabilly (expired), the subject matter of which are hereby incorporated by reference as if fully set forth herein. In one aspect, DNA encoding the FOXC1 monoclonal antibodies described herein can be isolated and sequenced using procedures known in the art (e.g., by using oligonucleotide probes that are capable of binding specifically to genes encoding the heavy and light chains of murine antibodies). In one embodiment, hybridoma cells may serve as a source of such DNA. Once isolated, the DNA can be placed into expression vectors, which are then transfected into host cells (e.g., simian COS cells, Chinese hamster ovary (CHO) cells, or myeloma cells that do not otherwise produce immunoglobulin protein) which synthesize the monoclonal antibodies. The DNA also can be modified, for example, by substituting the coding sequence for human heavy and light chain constant domains in place of the homologous murine sequences or by covalently joining all or part of a coding sequence for a non-immunoglobulin polypeptide to the immunoglobulin coding sequence. Such a non-immunoglobulin polypeptide can be substituted for the constant domains of an antibody of the invention, or can be substituted for the variable domains of one antigen-combining site of an antibody of the invention to create a chimeric bivalent antibody.

Antibody fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

In some embodiments, conservative variants of the antibodies may be produced. Such conservative variants employed in antibodies or functional antibody fragments, should retain amino acid residues that are necessary for correct folding and stabilizing between the VH and the VL regions, and will retain the charge characteristics of the residues in order to preserve the low isoelectric point (pi) and low toxicity of the molecules. Amino acid substitutions may be made in the VH and the VL regions to increase yield. Conservative amino acid substitution tables providing functionally similar amino acids are known to one of ordinary skill in the art. Generally, conservative variants will bind the target antigen with an equal to or greater efficiency than the parent monoclonal antibody.

According to the embodiments described herein, once the amino acid sequence of an antibody that is specific to a target antigenic peptide sequence of human FOXC1 such as those described above can be determined and a nucleic acid sequence encoding the amino acid sequence can be engineered. In addition, a variety of clones containing functionally equivalent nucleic acid molecules may be constructed, such as nucleic acid molecules which differ in sequence but which encode the same effect or molecule or antibody sequence. Thus, nucleic acids encoding FOXC1-specific antibodies or functional fragments thereof (such as those specific for the epitope AHAEQYPGGMARAYGPYTPQPQPKD, amino acids 51 to 75 of SEQ ID NO:1), conjugates and fusion proteins are also provided.

Anti-FOXC1 Derivatives and Conjugates

In some embodiments, the FOXC1 antibodies, modified antibodies or functional antibody fragments may include antibody derivatives that are chemically modified. For example, the antibody derivatives include, but are not limited to, antibodies that have been modified by glycosylation, acetylation, pegylation, phosphorylation, amidation or derivatization by known protecting/blocking groups, proteolytic cleavage, and linkage to a cellular ligand or other protein. Any of numerous chemical modifications may be carried out by known techniques, including, but not limited to, specific chemical cleavage, acetylation, gormylation and metabolic synthesis of tunicamycin. Additionally, the derivative may contain one or more non-natural amino acids.

In other embodiments, the FOXC1 antibody or functional antibody fragment may be conjugated to another substance to form an anti-FOXC1 conjugate. The anti-FOXC1 conjugates described herein can be prepared by known methods of linking antibodies with lipids, carbohydrates, proteins or other atoms and molecules. In one aspect, the anti-FOXC1 conjugate is formed by site-specific conjugation using a suitable linkage or bond. Site-specific conjugation is more likely to preserve the binding activity of an antibody or functional antibody fragment. The substance may be conjugated or attached at the hinge region of a reduced antibody component or antibody fragment via disulfide bond formation. For example, introduction of cysteine residues at the C-terminus of an scFv fragment, such as those introduce in the cys-diabodies described above, allows site-specific thiol-reactive coupling at a site away from the antigen binding site to a wide variety of agents. Alternatively, other linkages or bonds used to form the anti-FOXC1 conjugate may include, but is not limited to, a covalent bond, a non-covalent bond, a sulfide linkage, a hydrazone linkage, a hydrazine linkage, an ester linkage, an amido linkage, and amino linkage, an imino linkage, a thiosemicabazone linkage, a semicarbazone linkage, an oxime linkage and a carbon-carbon linkage.

In one embodiment, the anti-FOXC1 conjugate may include a FOXC1 antibody or functional FOXC1 antibody fragment conjugated to an intracellular delivery agent. In one aspect, the delivery agent is a cell penetrating peptide. Cell penetrating peptides are short peptides that facilitate cellular uptake of various molecules and substances (e.g., small molecules, peptides, proteins and nucleic acids), for delivery of such molecules and substances to the cytoplasm of a cell. This is accomplished by exploiting various cellular processes such as direct plasma membrane penetration, endocytosis mediated transport and translocation through formation of a transitory structure. Cell penetrating peptides that may be used to form an anti-FOXC1 conjugate for intracellular delivery include, but are not limited to, Trans-Activator of Transcription (TAT) protein (Mie et al. 2003; Wadia et al. 2004; Kameyama et al. 2006), Protein essential during penetration 1 (Pep-1) (Morris et al. 2001). Other delivery agents that may be used to deliver the FOXC1 antibody or functional fragment therein include, but are not limited to, cationic lipids (Weill et al. 2008), poly-L-arginine (Chen & Erlanger 2002); liposomal drugs (Roth et al. 2007) and polymeric carriers (e.g., poly(propyl acrylic acid; PPAA) (Stayton et al. 2005).

In one embodiment, the anti-FOXC1 conjugate may include a FOXC1 antibody or functional FOXC1 antibody fragment conjugated to a diagnostic agent. A “diagnostic agent” is an atom, molecule, compound or other substance that is useful in diagnosing, detecting or visualizing cancer or other conditions associated with FOXC1 by in vitro, ex vivo or in vivo methods known in the art and described below. According to the embodiments described herein, diagnostic agents may include, but are not limited to, radioactive substances (e.g., radioisotopes, radionuclides, radiolabels or radiotracers), dyes, contrast agents, fluorescent compounds or molecules, bioluminescent compounds or molecules, enzymes and enhancing agents (e.g., paramagnetic ions). In addition, it should be noted that some nanoparticles, for example quantum dots and metal nanoparticles (described below) may also be suitable for use as a detection agent.

Radioactive substances that may be used as diagnostic agents in accordance with the embodiments of the disclosure include, but are not limited to, ¹⁸F, ³²P, ³³P, ⁴⁵Ti, ⁴⁷Sc, ⁵²Fe, 59Fe, ⁶²Cu, ⁶⁴Cu, ⁶⁷Cu, ⁶⁷Ga, ⁶⁸Ga, ⁷⁵Sc, ⁷⁷As, ⁸⁶Y, ⁸⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as diagnostic agents in accordance with the embodiments of the disclosure include, but are not limited to, ions of transition and lanthanide metals (e.g. metals having atomic numbers of 6 to 9, 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

When the diagnostic agent is a radioactive metal or paramagnetic ion, the agent may be reacted with a reagent having a long tail with one or more chelating groups attached to the long tail for binding these ions. The long tail may be a polymer such as a polylysine, polysaccharide, or other derivatized or derivatizable chain having pendant groups to which may be bound to a chelating group for binding the ions. Examples of chelating groups that may be used according to the disclosure include, but are not limited to, ethylenediaminetetraacetic acid (EDTA), diethylenetriaminepentaacetic acid (DTPA), DOTA, NOTA, NETA, porphyrins, polyamines, crown ethers, bis-thiosemicarbazones, polyoximes, and like groups. The chelate is normally linked to the FOXC1 antibody or functional antibody fragment by a group which enables formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antibodies and carriers described herein. Macrocyclic chelates such as NOTA, DOTA, and TETA are of use with a variety of metals and radiometals including, but not limited to, radionuclides of gallium, yttrium and copper, respectively. Other ring-type chelates such as macrocyclic polyethers, which are of interest for stably binding nuclides, such as ²²³Ra for RAIT may be used. In certain embodiments, chelating moieties may be used to attach a PET imaging agent, such as an Al-¹⁸F complex, to a targeting molecule for use in PET analysis.

Contrast agents that may be used as diagnostic agents in accordance with the embodiments of the disclosure include, but are not limited to, barium, diatrizoate, ethiodized oil, gallium citrate, iocarmic acid, iocetamic acid, iodamide, iodipamide, iodoxamic acid, iogulamide, iohexyl, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, thallous chloride, or combinations thereof.

Bioluminescent and fluorescent compounds or molecules and dyes that may be used as diagnostic agents in accordance with the embodiments of the disclosure include, but are not limited to, fluorescein, fluorescein isothiocyanate (FITC), 5-dimethylamine-1-napthalenesulfonyl chloride, 4′,6-diamidino-2-phenylindole (DAPI), Oregon Green™, rhodamine, Texas red, tetrarhodimine isothiocynate (TRITC), Cy3, Cy5, lanthanide phosphors, green fluorescent protein (GFP), Yellow fluorescent protein (YFP), phycoerythrin and autoquenched fluorescent compounds that are activated by tumor-associated proteases and enzymes (e.g., luciferase, horseradish peroxidase, alkaline phosphatase, nanoparticles, biotin, digoxigenin) or a combination thereof.

Enzymes that may be used as diagnostic agents in accordance with the embodiments of the disclosure include, but are not limited to, horseradish peroxidase, alkaline phosphatase, acid phosphatase, glucose oxidase, β-galactosidase, β-glucoronidase or β-lactamase. Such enzymes may be used in combination with a chromogen, a fluorogenic compound or a luminogenic compound to generate a detectable signal.

In another embodiment, the anti-FOXC1 conjugate may include a FOXC1 antibody or functional FOXC1 antibody fragment conjugated to a therapeutic agent. A “therapeutic agent” as used herein is an atom, molecule, compound or other substance that is useful in the treatment of cancer or other conditions associated with FOXC1. Examples of therapeutic agents include, but are not limited to, drugs, chemotherapeutic agents, therapeutic antibodies and antibody fragments, toxins, radioisotopes, enzymes (e.g., enzymes to cleave prodrugs to a cytotoxic agent at the site of the tumor), nucleases, hormones, immunomodulators, antisense oligonucleotides, chelators, boron compounds, photoactive agents and dyes.

Chemotherapeutic agents are often cytotoxic or cytostatic in nature and may include alkylating agents, antimetabolites, anti-tumor antibiotics, topoisomerase inhibitors, mitotic inhibitors hormone therapy, targeted therapeutics and immunotherapeutics. In some embodiments the chemotherapeutic agents that may be used as therapeutic agents in accordance with the embodiments of the disclosure include, but are not limited to,13-cis-Retinoic Acid, 2-Chlorodeoxyadenosine, 5-Azacitidine, 5-Fluorouracil, 6-Mercaptopurine, 6-Thioguanine, actinomycin-D, adriamycin, aldesleukin, alemtuzumab, alitretinoin, all-transretinoic acid, alpha interferon, altretamine, amethopterin, amifostine, anagrelide, anastrozole, arabinosylcytosine, arsenic trioxide, amsacrine, aminocamptothecin, aminoglutethimide, asparaginase, azacytidine, bacillus calmette-guerin (BCG), bendamustine, bevacizumab, bexarotene, bicalutamide, bortezomib, bleomycin, busulfan, calcium leucovorin, citrovorum factor, capecitabine, canertinib, carboplatin, carmustine, cetuximab, chlorambucil, cisplatin, cladribine, cortisone, cyclophosphamide, cytarabine, darbepoetin alfa, dasatinib, daunomycin, decitabine, denileukin diftitox, dexamethasone, dexasone, dexrazoxane, dactinomycin, daunorubicin, decarbazine, docetaxel, doxorubicin, doxifluridine, eniluracil, epirubicin, epoetin alfa, erlotinib, everolimus, exemestane, estramustine, etoposide, filgrastim, fluoxymesterone, fulvestrant, flavopiridol, floxuridine, fludarabine, fluorouracil, flutamide, gefitinib, gemcitabine, gemtuzumab ozogamicin, goserelin, granulocyte-colony stimulating factor, granulocyte macrophage-colony stimulating factor, hexamethylmelamine, hydrocortisone hydroxyurea, ibritumomab, interferon alpha, interleukin-2, interleukin-11, isotretinoin, ixabepilone, idarubicin, imatinib mesylate, ifosfamide, irinotecan, lapatinib, lenalidomide, letrozole, leucovorin, leuprolide, liposomal Ara-C, lomustine, mechlorethamine, megestrol, melphalan, mercaptopurine, mesna, methotrexate, methylprednisolone, mitomycin C, mitotane, mitoxantrone, nelarabine, nilutamide, octreotide, oprelvekin, oxaliplatin, paclitaxel, pamidronate, pemetrexed, panitumumab, PEG Interferon, pegaspargase, pegfilgrastim, PEG-L-asparaginase, pentostatin, plicamycin, prednisolone, prednisone, procarbazine, raloxifene, rituximab, romiplostim, ralitrexed, sapacitabine, sargramostim, satraplatin, sorafenib, sunitinib, semustine, streptozocin, tamoxifen, tegafur, tegafur-uracil, temsirolimus, temozolamide, teniposide, thalidomide, thioguanine, thiotepa, topotecan, toremifene, tositumomab, trastuzumab, tretinoin, trimitrexate, alrubicin, vincristine, vinblastine, vindestine, vinorelbine, vorinostat, or zoledronic acid.

Therapeutic antibodies and functional fragments thereof, that may be used as diagnostic agents in accordance with the embodiments of the disclosure include, but are not limited to, alemtuzumab, bevacizumab, cetuximab, edrecolomab, gemtuzumab, ibritumomab tiuxetan, panitumumab, rituximab, tositumomab, and trastuzumab

Toxins that may be used as diagnostic agents in accordance with the embodiments of the disclosure include, but are not limited to, ricin, abrin, ribonuclease (RNase), DNase I, Staphylococcal enterotoxin-A, pokeweed antiviral protein, gelonin, diphtheria toxin, Pseudomonas exotoxin, and Pseudomonas endotoxin.

Radioisotopes that may be used as diagnostic agents in accordance with the embodiments of the disclosure include, but are not limited to, ³²P, ⁸⁹Sr, ⁹⁰Y. ^{99m}Tc, 99Mo, 131I, ¹⁵³Sm, ¹⁷⁷Lu, ¹⁸⁶Re, ²¹³Bi, ²²³Ra and ²²⁵Ac.

In another embodiment, the anti-FOXC1 conjugate may include a FOXC1 antibody or functional FOXC1 antibody fragment conjugated to a nanoparticle. The term “nanoparticle” refers to a microscopic particle, the size of which is measured in nanometers, e.g., a particle with at least one dimension less than about 100 nm. Nanoparticles are particularly useful as detectable substances because they are small enough to scatter visible light rather than absorb it. For example, gold nanoparticles possess significant visible light extinction properties and appear deep red to black in solution. As a result, compositions comprising FOXC1-specific antibody or fragments conjugated to nanoparticles can be used for the in vivo imaging of tumors or cancerous cells in a subject. At the small end of the size range, nanoparticles are often referred to as clusters. Metal, dielectric, and semiconductor nanoparticles have been formed, as well as hybrid structures (e.g. core-shell nanoparticles). Nanospheres, nanorods, and nanocups are just a few of the shapes that have been grown. Semiconductor quantum dots and nanocrystals are examples of additional types of nanoparticles. Such nanoscale particles, when conjugated to a FOXC1 antibody or functional antibody fragment, can be used as imaging agents for the in vivo detection of tumor cells as described above. Alternatively, nanoparticles can be used in therapeutic applications as drug carriers that, when conjugated to a PSCA-specific antibody or fragment of the present invention, deliver chemotherapeutic agents, hormonal therapeutic agents, radiotherapeutic agents, toxins, or any other cytotoxic or anti-cancer agent known in the art to cancerous cells that overexpress PSCA on the cell surface.

Any of the anti-FOXC1 conjugates described above may be further conjugated with one or more additional therapeutic agents, diagnostic agents, nanoparticles, carriers or a combination thereof. For example, a FOXC1 antibody or functional FOXC1 antibody fragment may be radiolabeled with ¹³¹I and conjugated to a lipid carrier, such that the anti-FOXC1-lipid conjugate forms a micelle. The micelle may incorporate one or more therapeutic or diagnostic agents. Alternatively, in addition to the carrier, the FOXC1 antibody or functional FOXC1 antibody fragment may be conjugated to ¹³¹I (e.g., at a tyrosine residue) and a drug (e.g., at the epsilon amino group of a lysine residue), and the carrier may incorporate an additional therapeutic or diagnostic agent.

Methods for diagnosing, clinically defining and prognosing cancer A FOXC1 antibody or functional FOXC1 antibody fragment such as those described above may be used to target a FOXC1 positive cell, such as cancer cells that express or overexpress FOXC1. FOXC1 expression has been shown to define biologically and clinically aggressive subsets in several types of cancer and may be used both as a diagnostic as well as prognostic biomarker. FOXC1 is also a suitable therapeutic target for cancer, as describe further below. Cancers that are associated with FOXC1 expression may include, but are not limited to, brain tumors (such as glioblastoma multiforme, or astrocytoma), breast cancer, colon cancer, gastric cancer, leukemia, melanoma, neuroendocrine tumors, prostate cancer (e.g., androgen-resistant prostate cancer), renal cell cancer and sarcomas (such as synovial sarcoma).

In particular, FOXC1 expression is an important theranostic biomarker that may be used to clinically define, diagnose prognose and treat primary and metastatic basal-like breast cancer and related basal-like subtypes. The relationship between FOXC1 expression and its role in basal-like breast cancer is described in detail in International Patent Application No. PCT/US10/44817, filed Aug. 6, 2010, the subject matter of which is hereby incorporated by reference as if fully set forth herein. Briefly, FOXC1 is elevated only in basal-like molecular subtypes of breast cancers, and has been demonstrated to be of high prognostic significance, as it is predictive of the high mortality and metastasis rate specifically associated with basal-like breast cancers.

Thus, in some embodiments, a FOXC1 antibody or functional antibody fragment as described above may be used in methods for diagnosing, clinically defining and prognosing primary and metastatic basal-like breast cancer and related basal-like subtypes. In one embodiment, the FOXC1 antibody, or a functional FOXC1 antibody fragment is a FOXC1 monoclonal antibody that is produced by a hybridoma cell line and specifically binds to a target antigenic peptide sequence (e.g., SEQ ID NOs:2-3) as described herein.

The FOXC1 antibody or functional antibody fragment described herein may be used to detect cells that express or overexpress FOXC1. This is accomplished by contacting one or more breast cancer cells in a biological sample with the FOXC1 antibody or functional antibody fragment. In some embodiments, the biological sample may by any biological sample that contains or is suspected of containing breast cancer cells including, but not limited to primary or metastatic tumors, blood, serum, plasma, lymph, lymph nodes, cerebrospinal fluid, bone marrow, interstitial fluid, and urine. In some embodiments, the biological sample may be removed from a subject having breast cancer or suspected of having cancer. In other embodiments, the biological sample containing or suspected of containing breast cancer cells may remain within the subject having breast cancer or suspected of having breast cancer, and may be detected by in vivo methods described further below.

In one embodiment, the FOXC1 antibody or functional antibody fragment may be conjugated to a diagnostic agent to allow for detecting the presence or absence of FOXC1 in the breast cancer cells. The diagnostic agent may be any suitable radioactive substance, dyes, contrast agent, fluorescent compound or molecule, bioluminescent compound or molecule, enzyme or enhancing agent described in detail above or otherwise known in the art. The diagnostic anti-FOXC1 conjugate may be conjugated to or associated with one or more additional substances described herein, such as a therapeutic anti-FOXC1 conjugate (as described below), unconjugated therapeutic agents, contrast solutions, carrier lipids or nanoparticles.

The detection of FOXC1 may be by any suitable in vitro or in vivo method. In one embodiment, an in vitro diagnostic or prognostic immunoassay will be performed to determine the expression level of FOXC1 in a tissue sample that contains cancer cells (e.g., breast cancer cells). According to some embodiments, the tissue sample may be taken from a subject having or suspected of having a cancer associated with FOXC1 (e.g. breast cancer) and compared to a normal (i.e., non cancerous) or control tissue sample (i.e., known cancerous or benign tissue sample). In other embodiments, the tissue sample may be assayed for the presence or absence of FOXC1.

Detecting the presence or absence of FOXC1 or detecting an expression level of FOXC1 in a tissue sample is accomplished by any suitable in vitro immunoassay such as immunohistochemistry, immunocytochemistry, fluorescent in situ hybridization (FISH) or other immunofluorescence staining and Western blots. Because plasma membranes are generally not permeable to antibodies, proteins, peptides, drugs and other therapeutic substances, in vitro immunoassays that use the contents of whole cells or whole cell preparations (e.g., immunocytochemistry, immunofluorescence staining, Western blot) should include a step to lyse or permeablize the cells in the tissue sample to allow the FOXC1 antibody or functional antibody fragment to reach its intracellular target, FOXC1.

In another embodiment, an in vivo imaging method is used to detect the presence or absence of FOXC1 in a subject having or suspected of having a cancer associated with FOXC1 (e.g. breast cancer) to visualize the target cells within the topography of the subject's body. The in vivo imaging method may include administering the FOXC1 antibody or functional antibody fragment to the subject and exposing the subject to an imaging modality able to detect the diagnostic agent that is conjugated to the antibody or functional fragment. Suitable imaging modalities that may be used in accordance with the methods described herein include, but are not limited to, magnetic resonance imaging (MRI), positron emission tomography (PET) or microPET, computed tomography (CT), PET/CT combination imager, cooled charged coupled device (CCD), camera optical imaging, optical imaging (e.g., bioluminescent optical imaging, fluorescent optical imaging, or absorption of reflectance) and single photon emission computed tomography (SPECT).

Cellular plasma membranes are generally not permeable to antibodies, proteins, peptides, drugs and other therapeutic substances. Therefore, when an in vivo method is used, the diagnostic anti-FOXC1 conjugate must be delivered intracellularly. According to some embodiments, the anti-FOXC1 conjugate may be delivered intracellularly by conjugation to or association with an intracellular delivery agent or a carrier. In one embodiment, the delivery agent or carrier may include a cell penetrating peptide or a delivery reagent. Cell penetrating peptides that may be used to form an anti-FOXC1 conjugate for intracellular delivery include, but are not limited to, TAT protein and Pep-1 protein. Other delivery agents that may be used to deliver the FOXC1 antibody or functional fragment therein include, but are not limited to, nanoparticles, cationic lipids, poly-L-arginine; liposomal drugs and polymeric carriers (e.g., poly(propyl acrylic acid) (PPAA). In other embodiments, the anti-FOXC1 conjugate may be delivered intracellularly by any other suitable method, for example, gene therapy, delivery of genetic material via viral vector, receptor mediated endocytosis and production of intrabodies,

In addition to diagnosing a cancer associated with FOXC1 expression, the anti-FOXC1 conjugate may also be used to stage, and monitor cancer progression according to method that are similar to those described above.

Methods for Treating Cancer

In some embodiments, methods for treating cancer or other conditions associated with overexpression of FOXC1 are provided. Such methods include administering to a subject a therapeutically effective amount of a pharmaceutical composition that includes a FOXC1 antibody, or a functional FOXC1 antibody fragment as described above. According to some embodiments, the FOXC1 antibody is a monoclonal antibody, a modified antibody or a functional antibody fragment as described in detail above.

When a FOXC1 antibody is administered as part of a pharmaceutical composition, it must be delivered intracellularly. As described above, intracellular delivery of the FOXC1 antibody may be accomplished by conjugation to or association with an intracellular delivery agent or a carrier or by any other suitable method known in the art. Thus, in some embodiments, the FOXC1 antibody or functional fragment is conjugated to an intracellular delivery agent to allow for delivery of the antibody to the cytoplasm of cells.

“Treating” or “treatment” of a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

A “therapeutically effective amount” or a “therapeutically effective dose” is an amount of a compound that produces a desired therapeutic effect in a subject, such as preventing or treating a target condition or alleviating symptoms associated with the condition. The precise therapeutically effective amount is an amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of a compound and adjusting the dosage accordingly. For additional guidance, see Remington: The Science and Practice of Pharmacy 21st Edition, Univ. of Sciences in Philadelphia (USIP), Lippincott Williams & Wilkins, Philadelphia, Pa., 2005, the subject matter of which is hereby incorporated by reference as if fully set forth herein.

In one embodiment, the pharmaceutical composition may include a FOXC1 antibody, modified antibody or a functional antibody fragment by itself or a therapeutic anti-FOXC1 conjugate. The conjugate may include a FOXC1 antibody, modified antibody or a functional antibody fragment conjugated to one or more therapeutic agents as described above. In one embodiment, the FOXC1 antibody, or a functional FOXC1 antibody fragment is a FOXC1 monoclonal antibody is produced by a hybridoma cell line and specifically binds to a target antigenic peptide sequence (e.g., SEQ ID NOs:2-3).

A FOXC1 antibody, modified antibody or a functional antibody fragment or a therapeutic anti-FOXC1 conjugate may be conjugated to or associated with one or more additional substances described herein, such as diagnostic anti-FOXC1 conjugates (described above), unconjugated diagnostic agents, contrast solutions, carrier lipids or nanoparticles.

In some embodiments, the pharmaceutical composition may also include a pharmaceutically acceptable carrier. A pharmaceutically acceptable carrier may be a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or some combination thereof. Each component of the carrier must be “pharmaceutically acceptable” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits.

The pharmaceutical compositions described herein may be administered by any suitable route of administration. A route of administration may refer to any administration pathway known in the art, including but not limited to aerosol, enteral, nasal, ophthalmic, oral, parenteral, rectal, transdermal (e.g., topical cream or ointment, patch), or vaginal. “Transdermal” administration may be accomplished using a topical cream or ointment or by means of a transdermal patch. “Parenteral” refers to a route of administration that is generally associated with injection, including infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal.

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLE 1

### Generation of a FOXC1 Monoclonal Antibody

An anti-FOXC1 antibody was generated as described below.

Antigenic peptide sequence design. An antigenic peptide sequence was designed using the Lasergene software suite by DNASTAR™. The software was used to determine the regions of the FOXC1 protein that maximize hydrophilicity (H), antigenicity (A), and surface probability (SP), but do not contain turns or glycosylation sites. In addition, BLAST searches against gene databases were also performed to ensure that the homology of these peptides with other FOX family genes is low.

Based on the results of the design process described above, residues 51-75 of the human FOXC1 protein were selected as the antigenic peptide sequence as shown below:

5′-AHAEQYPGGMARAYGPYTPQPQPKD-3′ (SEQ ID NO:2)

A cysteine residue was added to the N-terminus (shown below) to assist in conjugation to the carrier protein as necessary.

5′-C-AHAEQYPGGMARAYGPYTPQPQPKD-3′ (SEQ ID NO:3)

This antigenic peptide sequence is found in the FOXC1 N-terminal transactivation domain. Previous reports have suggested that the FOXC1 N-terminal domain plays an important role in the function of FOXC1 in transcriptional activation (Berry et al., 2002). Therefore, FOXC1 antibodies that specifically bind to this domain are not only useful in diagnostic and prognostic assays, but are likely to be good candidates for treating basal like breast cancers and other cancers that are associated with the expression or overexpression of FOXC because they block the activity of FOXC1.

Production of hybridoma and antibodies. Hybridoma and antibody generation was contracted through Pro-Sci Inc. Briefly, Balb/c mice were injected with the FOXC1 peptide antigen sequence described above to generate lymphocytes that produce or are able to produce a complementary monoclonal antibody. Next, immunized mice with high titers of anti-FOXC1 antibody response and positive immunoblot signals were selected by ELISA and immunoblotting,

The hybridoma cell line B2E3 was then generated by fusing myeloma cells with B cells isolated from the spleen of the selected immunized mice. Then ELISA and immunoblotting were used to screen for the positive hybridoma cell clones, followed by two consecutive subclonings. Based on immunoblotting results, several positive clones including the hybridoma cell line B2E3 were expanded in culture. The antibodies produced by the clones were purified using an IgG purification column.

The antibodies produced by the hybridomas as described above may be used for detection of FOXC1 protein and for coupling other molecules to FOXC1 protein. Further, the anti-FOXC1 antibody was tested by immunoblotting, immunofluorescence, and immunohistochemistry as described below.

Immunoblotting. MCF-7 human breast cancer cells were transfected with FOXC1(+) or FOXC1(−) vectors. Whole cell lysates for western blotting were generated by cell lysis buffer (50 mM Tris-HCl, pH 7.4, 150 mM NaCl, 2 mM EDTA, 1% NP-40, 10% glycerol) supplemented with a protease inhibitor cocktail (Sigma, St Louis, Mo.). Equal amounts of protein were separated by 10% SDS-PAGE and then transferred onto a nitrocellulose membrane. The remaining steps were conducted according to a standard immunoblotting protocol (Qu et al., 2009). Immunoblotting was done with polyclonal commercial FOXC1 antibodies (Santa Cruz Biotechnology), and FOXC1 monoclonal antibodies isolated from hybridoma cell line B2E3. After the primary antibody incubation, the membrane was again washed with PBST three times (5 min each) and then incubated with a horseradish peroxidase (HRP)-linked secondary antibody (Amersham, Piscataway, N.J.) at a dilution of 1:4000 in blocking solution. The membrane was washed and bands were visualized using chemiluminescence assays.

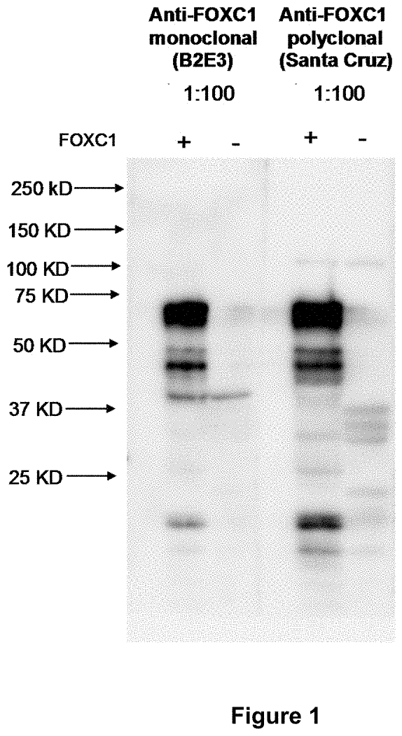

As shown in FIG. 1, the FOXC1 monoclonal antibody isolated from hybridoma cell line B2E3 produced fewer non-specific bands as compared to a polyclonal commercial anti-FOXC1 antibody from Santa Cruz Biotechnology Inc. Other available commercial anti-FOXC1 antibodies would produce similar or less desirable results compared to the Santa Cruz anti-FOXC1 antibody in terms of specificity and sensitivity. Therefore, the FOXC1 antibody described herein is more sensitive and is superior as a diagnostic and prognostic tool than antibodies that are currently available.

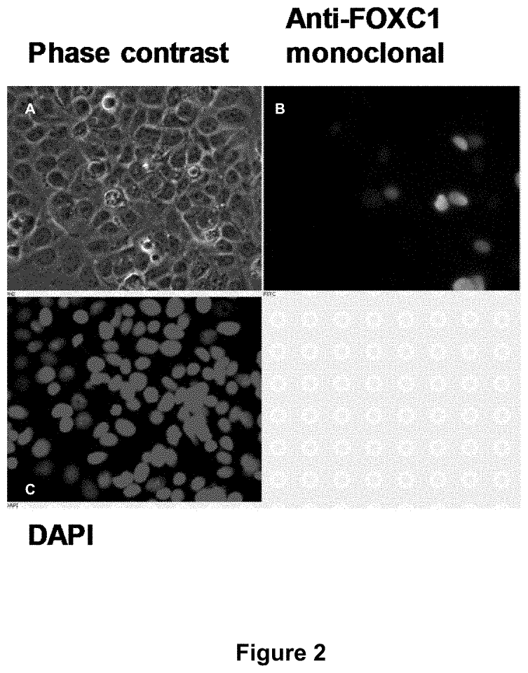

Immunofluorescence staining. MCF-7 cells were transiently transfected with GFP-FOXC1 plasmid for 24 h. Then the cells were then digested with trypsin and seeded in chamber slides (BD Biosciences, Franklin Lakes, N.J.) (FIG. 2A). After 12-h incubation, cells were fixed with 4% formaldehyde and then permeabilized with PBS containing 0.1% Triton X-100. Slides were blocked by 5% BSA for 30 minutes and incubated with a primary FOXC1 monoclonal antibody isolated from hybridoma cell line B2E3 at room temperature for 1 h. Cells were then incubated with an Alexa Fluor 488—conjugated secondary antibody (1:200, Invitrogen) for 30 min (FIG. 2B). Slides were washed by PBS three times for 5 minutes each, mounted in DAPI, and observed under a high resolution Nikon TI-E microscope (FIG. 2C). The results of this experiment are shown in FIG. 2.

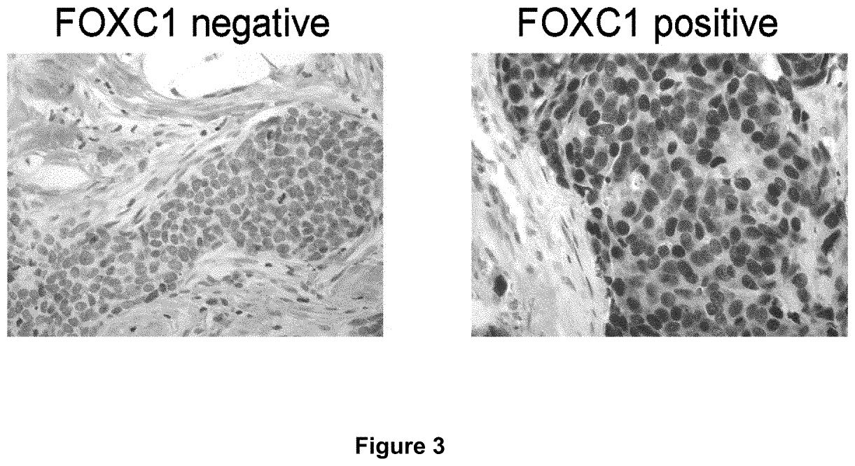

Immunohistochemistry. Immunohistochemistry (IHC) was performed according to standard protocols, with the exception of: (1) antigen retrieval was performed for 30 minutes at 100° C. in citrate buffer(pH 6.0); (2) primary mouse anti-FOXC1 antibodies were allowed to hybridize for 16 hrs at 4 C (overnight); and (3) the labeling reagent used was a polymer-based anti-mouse antibody (BioCare) with DAB chromogen. The presence of FOXC1 in breast cancer tissues (FOXC1+and FOXC1−) tissues is shown in FIG. 3.

### REFERENCES

The references, patents and published patent applications listed below, and all references cited in the specification above are hereby incorporated by reference in their entirety, as if fully set forth herein.

Berry F B, Saleem R A, Walter M A (2002). “FOXC1 transcriptional regulation is mediated by N- and C-terminal activation domains and contains a phosphorylated transcriptional inhibitory domain.”. J. Biol. Chem. 277 (12): 10292-7.

Chen B X, Erlanger B F, Intracellular delivery of monoclonal antibodies, Immunology Letters (2002) 84(1):63-67.

Huson P. J. & Souriau C., Engineered Antibodies, Nature Medicine (2003) 9(1):129-134.

Kameyama S, Horie M, Kikuchi T, Omura T, Takeuchi T, Nakase I, Sugiura Y and Futaki S, Bioconjugate Chem., 2006, 17, 597-602.

Köhler, G. & Milstein, C., Continuous cultures of fused cells secreting antibody of predefined specificity.’ Nature 256, 495-497 (1975).

Mie M, Takahashi F, Funabashi H, Yanagida Y, Aizawa M and Kobatake E, Intracellular delivery of antibodies using TAT fusion protein A (2003) 310(3):730-734.

Morris M C, Depollier J, Mery J, Heitz F and Divita G, Nat. Biotechnol., 2001, 19, 1173-1176.

Nishimura D Y, Swiderski R E, Alward W L, Searby C C, Patil S R, Bennet S R et al (1998). The forkhead transcription factor gene FKHL7 is responsible for glaucoma phenotypes which map to 6p25. Nat Genet 19: 140-7.

Qu Y, Wang J, Sim M S, Liu B, Giuliano A, Barsoum J et al (2009). Elesclomol, counteracted by Akt survival signaling, enhances the apoptotic effect of chemotherapy drugs in breast cancer cells. Breast Cancer Res Treat.

Ray P, Wang J, Qu Y, Sim M, Shamonki J, Bagaria S P et al (2010). FOXC1 Is a Potential Prognostic Biomarker with Functional Significance in Basal-like Breast Cancer. Cancer Research May 15. Ray et al. 2010, Annals of Surgical Oncology, under review.

Ray P, Wang J, Qu Y, Sim M, Shamonki J, Bagaria S P et al (2010). FOXC1 Is a Potential Prognostic Biomarker with Functional Significance in Basal-like Breast Cancer. Cancer Research May 15.

Roth A, Drummond D C, Conrad F, Hayes M E, Kirpotin D B, Benz C C, Marks J D and Liu B, Anti-CD166 single chain antibody-nediated intracellular delivery of liposomal drugs to prostate cancer cells. Molecular Cancer Therapy 2007; 6(10):2737-46.

Stayton P S, El-Sayed M E, Murthy N, Bulmus V, Lackey C, Cheung C, Hoffman A S, ‘Smart’ delivery systems for biomolecular therapeutics, Orthod Craniofac Res. 2005; 8(3):219-25.

Wadia J S, Stan R V, Dowdy S F: Transducible TAT-HA fusogenic peptide enhances escape of TAT-fusion proteins after lipid raft macropinocytosis. Nat Med 2004, 10:310-315.

Weill C O, Biri S, Erbacher P, Cationic lipid-mediated intracellular delivery of antibodies into live cells. BioTechniques 44(7) vii-xi (2008).

1-20. (canceled)

21. A method of diagnosing a basal-like breast cancer in a subject having breast cancer or suspected of having breast cancer, the method comprising:
contacting one or more breast cancer cells in a biological sample with an antibody or functional fragment thereof, wherein the antibody or functional fragment thereof binds an antigenic peptide sequence that is part of human FOXC1;
detecting the presence or absence of FOXC1 in the one or more breast cancer cells; and
diagnosing the subject as having a basal-like breast cancer when FOXC1 is present,
wherein the antigenic peptide sequence is:

| | |
|---|---|
| 5′-AHAEQYPGGMARAYGPYTPQPQPKD-3′; | (SEQ ID NO: 2) |
| or | |
| 5′-C-AHAEQYPGGMARAYGPYTPQPQPKD-3′. | (SEQ ID NO: 3) |

22. The antibody or functional fragment of claim 21, wherein the antibody or functional fragment thereof is a monoclonal antibody.

23. The method of claim 21, wherein the biological sample is a primary or metastatic tumor sample, a blood sample, a serum sample, a plasma sample, a lymph sample, a lymph node sample, a cerebrospinal fluid sample, a bone marrow sample, an interstitial fluid sample or a urine sample.

24. The method of claim 21, wherein the antibody or functional fragment thereof is conjugated to a diagnostic agent.

25. The method of claim 24, wherein the diagnostic agent is a radioactive substance, dyes contrast agent, fluorescent compound or molecule, bioluminescent compound or molecule, enzyme or enhancing agent.

26. The method of claim 21, wherein detecting the presence or absence of FOXC1 is accomplished by an in vitro immunoassay.

27. The method of claim 26, wherein the in vitro immunoassay is immunocytochemistry (ICC), immunohistochemistry (IHC), Western blot or fluorescent in situ hybridization (FISH).

28. The method of claim 21, further comprising:
lysing or permeabilizing the one or more breast cancer cells when the in vitro immunoassay uses the contents of whole cells or whole cell preparations.


SEQUENCE LISTING (3 sequences)

SEQ ID NO: 1 - protein, 553 aa, Homo sapiens
  ref <308>: DATABASE ACCESSION NUMBER: GenBank/AAH70124
  ref <309>: DATABASE ENTRY DATE: 2006-07-15
  ref <313>: RELEVANT RESIDUES IN SEQ ID NO: (1)..(553)
MQARYSVSSPNSLGVVPYLGGEQSYYRAAAAAAGGGYTAMPAPMSVYSHPAHAEQYPGGMARAYGPYTPQPQPKDMVKPPYSYIALITMAIQNAPDKKITLNGIYQFIMDRFPFYRDNKQGWQNSIRHNLSLNECFVKVPRDDKKPGKGSYWTLDPDSYNMFENGSFLRRRRRFKKKDAVKDKEEKDRLHLKEPPPPGRQPPPAPPEQADGNAPGPQPPPVRIQDIKTENGTCPSPPQPLSPAAALGSGSAAAVPKIESPDSSSSSLSSGSSPPGSLPSARPLSLDGADSAPPPPAPSAPPPHHSQGFSVDNIMTSLRGSPQSAAAELSSGLLASAAASSRAGIAPPLALGAYSPGQSSLYSSPCSQTSSAGSSGGGGGGAGAAGGAGGAGTYHCNLQAMSLYAAGERGGHLQGAPGGAGGSAVDNPLPDYSLPPVTSSSSSSLSHGGGGGGGGGGQEAGHHPAAHQGRLTSWYLNQAGGDLGHLASAAAAAAAAGYPGQQQNFHSVREMFESQRIGLNNSPVNGNSSCQMAFPSSQSLYRTSGAFVYDCSKF

SEQ ID NO: 2 - protein, 25 aa, Homo sapiens
AHAEQYPGGMARAYGPYTPQPQPKD

SEQ ID NO: 3 - protein, 26 aa, Homo sapiens
CAHAEQYPGGMARAYGPYTPQPQPKD